(19) United States
(12) Patent Application Publication
(10) Pub. No.: US 2026/0014287 A1
(43) Pub. Date: Jan. 15, 2026

(54) LIQUID DISPENSER

(71) Applicant: Fast & Fluid Managment B.V., Sassenheim (NL)

(72) Inventors: Mark Johannes BAAK, Sassenheim (NL); Bob Leonard HONSBEEK, Sassenheim (NL); Marcel Hendrikus Petrus ENGELS, Sassenheim (NL)

(73) Assignee: Fast & Fluid Managment B.V., Sassenheim (NL)

(21) Appl. No.: 19/137,907

(22) PCT Filed: Nov. 28, 2023
(86) PCT No.: PCT/EP2023/083418

§ 371 Date: Jun. 11, 2025

(30) Foreign Application Priority Data
Dec. 14, 2022 (EP) 22213521.2

(51) Int. Cl. A61L 2/10

(52) U.S. Cl. CPC A61L 2/10; A61L 2202/23

(57) ABSTRACT

Dispenser comprising at least one container and at least one source for biocidal radiation, such as a UV-light source. The at least one container comprises a lid, which is transparent for the biocidal radiation. The source for biocidal radiation is at a distance above the lid, e.g., in the dispenser housing.

### CROSS-REFERENCE TO RELATED APPLICATIONS

This application is a national phase application of International Application No. PCT/EP2023/083418, filed Nov. 28, 2023, which claims priority to European Patent Application Serial No. 22213521.2, filed Dec. 14, 2022, all of which are incorporated herein by reference.

The present disclosure relates to a liquid dispenser comprising at least one container for holding a liquid to be dispensed, such as a water borne emulsion or dispersion, in particular a pigment dispersion, e.g., a paint colorant or tinting paste. Such dispensers allow to dispense and mix selected colorants, optionally with a selected base paint, in accordance with a pre-defined formulation of a desired paint colour, for instance at a point of sale or a car refinish body shop.

The dispenser typically comprises a support for carrying the containers, such as a turntable or a static platform, and a dispenser housing.

The containers or canisters typically comprise a dispense nozzle, e.g., at a bottom side of the container, and a refill opening, e.g., at a headspace section of the container. The container may comprise a pump, or can selectively be connected to a pump to dispense a selected amount of the tinting paste in response to a control unit. Optionally, the containers are provided with a stirrer.

Due to environmental regulations, present day paints and tinting pastes are usually water borne. Such aqueous compositions are sensitive for microbiological activity, in particular growth of mould, algae, bacteria or other microorganisms. This is particularly problematic for tinting pastes, which are typically stored for longer periods in containers where only occasionally small amounts are dispensed from. Microbiological fouling and mildew can particularly occur in the headspace of such containers or at the dispensing nozzle.

EP 3 395 454 A1 and EP 3 854 473 A1 teach to use UV-light to inhibit microbial activity within the containers. In EP 3 854 473 A1, one or more UV-sources are integrated in the lid of the containers. This is relatively expensive. Powering of the UV sources is complicated and requires wiring to each container, usually on a rotating turntable. Relative small UV-LED's can be used with only limited power.

It is an object of the invention to provide a dispenser with biocidal radiation sources which are easier to control in a more efficient way.

The object of the invention is achieved with a dispenser comprising at least one container and at least one source for biocidal radiation, such as a UV-light source. The container comprises a lid, which is transparent for the applied biocidal radiation. The source of biocidal radiation is at a distance above the lid, e.g., in the dispenser housing. This way, a single system can be used centrally for all containers instead of using a separate system for each container. A central electronic circuitry can be used with less wiring, which does not have to cross moving parts, such as a turntable. Radiation sources with higher power can be used, compared to the UV-LED's of EP 3 854 473 A1. Moreover, it is easier to test proper functioning of a single system compared to testing separate UV sources for each individual container.

The source for biocidal radiation can for example be arranged to irradiate the surface of a colorant in the container and/or an inner wall of a headspace of the container and/or a stirrer within the container.

Suitable biocidal radiation is for example UV-light, e.g., UVC and/or UVB light, e.g., UV light having a wave length of 100-320 nm. Such short-wave UV light disrupts DNA base pairing, causing inactivation of bacteria, viruses, fungi and protozoans. Alternatively, the biocidal radiation can be microwave radiation, x-ray, infrared or other suitable type of biocidal radiation. Combinations of different types of radiation can also be used.

The use of shorter wave lengths, e.g., 150-200 nm, in particular about 185 nm, generates ozone, which has an additional sanitizing effect.

Good results are obtained if biocidal radiation, in particular UVB and/or UVC-light, is irradiated with an intensity of at least 4 mJ/cm2, e.g., at least 8 mJ/cm2, e.g., at least 10 mJ/cm2, e.g., at least 12 mJ/cm2.

The biocidal radiation can be irradiated, e.g., continuously, pulsewise or on demand, for example after temporary removal and subsequent replacement of the lid resulting in shorter or longer exposure of the headspace to ambient air and possible microbial contamination.

Optionally, the control unit can be configured to start biocidal radiation if the liquid level in the canister is below a set point and/or if microbial growth is detected.

Suitable UV-light sources are for instance high-or low-pressure mercury lamps, excimer lamps and UVC LED's.

The lids of the containers are transparent for the used biocidal radiation. A lid is considered to be transparent for biocidal radiation if an effective amount of the radiation passes the lid to reduce microbial activity, e.g., if more than 50%, e.g., more than 70% of the irradiance passes the lid. Such transparency depends on the type of material and the thickness of the lid. UV transparency of a lid can be determined as the ratio of the illuminance per unit area (e.g., expressed in lux) of a light source shielded by the lid and the illuminance per unit area of the same light source without shielding by the lid, times 100%. A lid is considered UV-transparent if the UV transparency is above 50%, e.g., at least 70%, e.g., at least 80%, e.g., at least 90%.

The UV-transparent lid can be made of a UV-transparent material, for example selected from the group of quartz glass, borosilicate glass, polystyrene, polymethyl methacrylate, polycarbonate or fluorinated ethylene propylene (FEP, or more particularly a copolymer of tetrafluoroethylene and hexafluoropropylene).

In a specific embodiment, the biocidal radiation source(s) can be moveable relative to the container(s) and/or the container(s) can be moveable relative to the biocidal radiation source(s). The dispenser may for example comprise a movable support, such as a turntable, supporting a plurality of the containers, wherein the support is movable relative to the biocidal radiation source. If a turntable is used, the containers can be arranged in concentric circular arrays and the source of biocidal radiation can be arranged to irradiate each container during time intervals of essentially the same length. Alternatively, the dispenser can have a static platform supporting the containers and having one or more sources of biocidal radiation movable along the container lids.

In a specific embodiment, the source of biocidal radiation can comprise a plurality of radiation devices, each circular array of containers comprising at least one of the radiation devices. By turning the turntable, each container passes at least one of the radiation devices. In such an arrangement, it is advantageous if each radiation device has a width which is proportional with the radius of the respective concentric array in order to ensure that all containers are irradiated during time intervals of the same length.

In a specific embodiment, the dispenser can have a control unit, for example configured to custom control irradiation of each individual container. Optionally, type, wave length, duration, intensity of the irradiation and/or further irradiation parameters can be selected dependent on one or more container parameters, such as liquid level within the container, composition of the liquid within the container, or the level of microbial activity in the container headspace. A lower liquid level in the container means a larger distance between the radiation source and the liquid level. Since the irradiation beam is typically divergent, a larger area will be irradiated if the liquid level in the container is low. To prevent underexposure of lower liquid levels, the control unit can be programmed to adjust intensity and/or duration of the irradiation depending on the liquid level. Optionally, the control unit can be configured to move the radiation source to scan the liquid surface and/or inner walls of the container headspace.

Optionally, each container is exposed to biocidal radiation for a time period determined per container in dependence of one or more parameters, such as liquid level in the container, detected microbial activity, composition of the liquid in the container, etc.

Optionally, the radiation devices are controlled independently of each other.

In a further embodiment, the source of biocidal radiation can be moveable, e.g., along a guide, in radial direction relative to the central axis of rotation of the turntable. The source of biocidal radiation can be controlled to irradiate each container with a customized level of irradiation or with the same level of irradiation, e.g., the same intensity and/or duration.

Optionally, the dispenser can comprise a fixture carrying a plurality of the radiation devices and having one or more openings allowing passage of radiation from the radiation devices to at least one of the containers positioned below the respective opening. In a specific embodiment, the radiation devices can be positioned above a single opening shaped as a circular segment coaxial with the turntable. As a result, containers of the outer array are exposed to the respective UV-light source for the same time period as the containers of the inner array. This way, UV-light sources of the same intensity can be used.

In a still further embodiment, the source of biocidal radiation comprises one or more reflectors, such as mirrors, to deflect a radiation beam from the source of biocidal radiation to a selected one of the containers. Optionally, the reflectors can be moveable or be shaped to focus reflected radiation in order to prevent part of the radiation from missing the targeted container.

The invention is further explained with reference to the accompanying drawings showing exemplary embodiments.

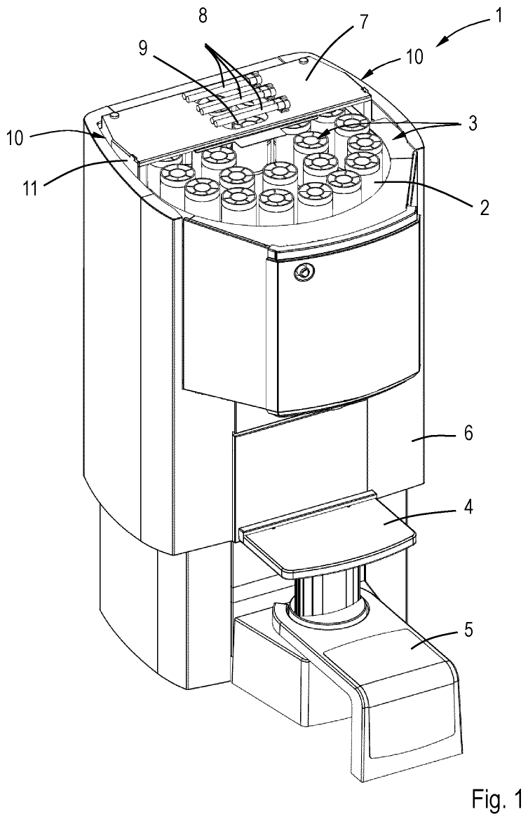

FIG. 1: shows a dispenser in perspective view;

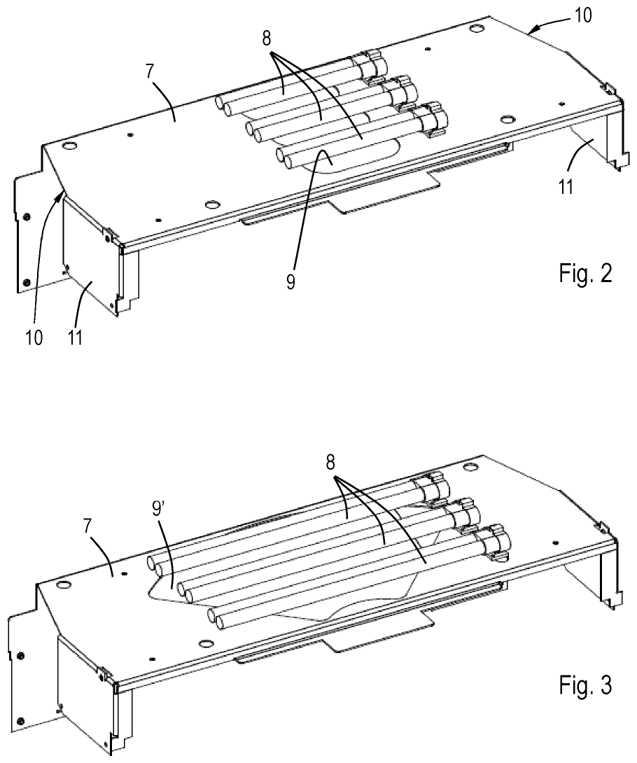

FIG. 2: shows a UV-light source fixture of the dispenser f FIG. 1;

FIG. 3: shows an alternative embodiment of a UV-light source fixture;

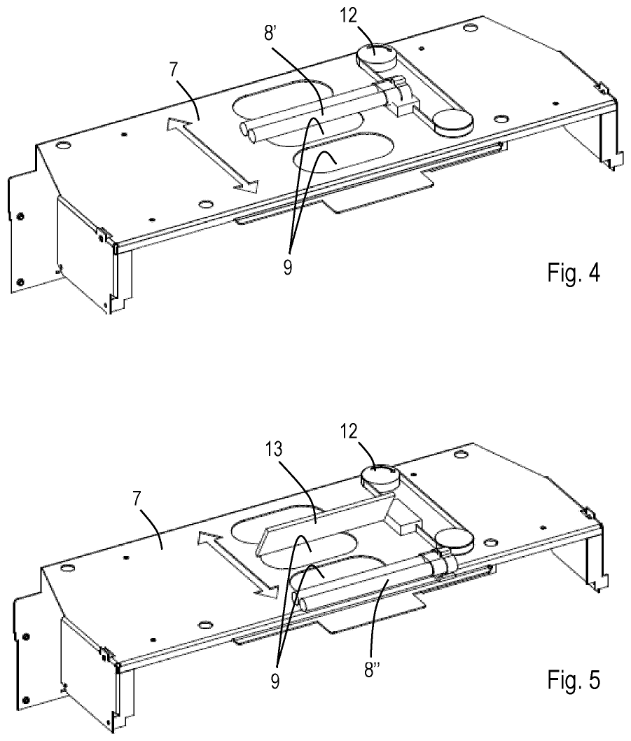

FIG. 4: shows a third embodiment of a UV-light source fixture;

FIG. 5: shows schematically a fourth embodiment.

FIG. 1 shows a dispenser 1 for mixing and dispensing paint products. The dispenser 1 comprises containers or canisters 2 arranged in three concentric circular arrays 3. Each container 2 holds a water borne tinting paste of a specific colour. The containers 2 are positioned on a turntable (not shown) which is coaxial with the circular arrays 3 of containers 2. The dispenser 1 further comprises a platform 4 for positioning a paint can or similar receptacle (not shown). When a user inputs a desired paint colour, a control unit determines a paint formulation matching the selected colour, e.g., from a database of paint formulations. The determined paint formulation consists of a tinting paste or mixture of tinting pastes available in the respective containers of the dispenser, and optionally a base paint. The turntable can be rotated to position a container 2 above the platform 4, so a tinting paste held in the selected container 2 can be dispensed into the paint can on the platform 4. The tinting pastes selected in accordance with the determined paint formulation can consecutively be dispensed and mixed.

The dispenser 1 also comprises a user interface 5, enabling an operator or user to input a desired paint colour. The control unit will sequentially position the containers 2 containing the required tinting pastes above the paint can on the platform 4 and control dispensing of the tinting pastes one by one from the respective containers 2. The dispenser 1 also comprises a housing 6 hiding the internals of the dispenser 1 from view, including the containers 2 and the turntable.

A fixture 7 supporting three UV-light sources 8 is positioned at a distance above the containers 2. The housing 6 comprises a cover (not shown), shielding the UV-light sources 8 and hiding the containers 2 from view. The fixture 7 is shown in more detail in FIG. 2 and comprises a substantially horizontal plate with three openings 9 and opposite side edges 10 provided with a flange 11 for connecting the fixture 7 to internal walls of the housing 6.

The three U-light sources 8 have a fixed position on the fixture 7. Each one of the UV light sources 8 is above one of the openings 9 and above one of the circular arrays 3 of containers 2. When the turntable rotates, the containers 2 pass the UV-light source 8 of the respective circular array 3 one by one, so each container 2 is irradiated via the respective opening 9 in the fixture 7 by the associated UV-light source 8. During rotation of the turntable, the UV-light sources 8 can irradiate continuously, or they can irradiate the containers pulsewise, sending one or more pulses when one of the containers passes by.

When the turntable rotates, the containers 2 of the outer circular array 3 move faster than the containers 2 of the middle and inner circular arrays 3. As a result, the containers 2 of the outer circular array 3, pass the respective UV-light source 8 in a shorter time period. To compensate for this, the UV-light source 8 for the outer circular array 3 of containers 2 can be controlled to irradiate the containers 2 with enhanced intensity, while the UV-light source 8 for the middle circular array 3 of containers 2 can be controlled to irradiate the containers 2 with normal intensity and the UV-light source 8 for the inner circular array 3 of containers 2 can be controlled to irradiate the containers with reduced intensity.

FIG. 3 shows an embodiment, where the fixture 7 has a single central opening 9′ shaped as a circular segment coaxial with the turntable. As a result, containers 2 of the outer circular array 3 of containers 2 are exposed to the respective UV-light source 8 during time periods of the same length as the containers 2 of the middle and inner circular arrays 3 of containers 2. This way, UV-light sources 8 of the same intensity can be used.

FIG. 4 shows a further embodiment, having the same fixture 7 as the embodiment of FIG. 2, with three openings 9 of the same size and shape but further having a single UV-light source 8′ connected to a driving mechanism 12 for moving the UV-light source 8′ along the row of the three openings 9. In a first position, the UV-light source 8′ is above the outer circular array of containers and above the respective opening 9 in the fixture 7. In a second position, the UV-light source 8 is above the middle circular array of containers and above the respective opening 9 in the fixture 7. In a third position, the UV-light source 2 is above the inner circular array of containers and above the respective opening 9 in the fixture 7.

FIG. 5 shows schematically a dispenser 1 comprising a UV-light source 8″ with a fixed position and a reflector 13 above three aligned openings 9 of the fixture 7. The reflector 13 is connected to a drive 12 which can move between positions above the respective openings 9. In each position, the reflector 13 deflects a UV-beam 14 from the UV-light source 8″ to a container positioned below the respective opening 9. The reflector 13 can be tiltable relative to the drive 12. The reflector 13 can be curved to focus or direct the beam from the UV-light source. Optionally, the reflector can be flexible with an curvature which is adjustable in response to the control unit so the beam can be focused, scattered or directed depending on parameters, such as the liquid level in the container. The reflector 13 can be a mirror or have a top layer which is nano-patterned to optimize light distribution.

The shown embodiments, are provided with three circular arrays of containers on the turntable. Alternatively, the dispenser can have only one circular array or containers, or it can have two, four or more circular arrays of containers. In a further alterative embodiment, the dispenser may have a static platform or a linear slider table allowing linear movement of the containers relative to the dispense opening and the biocidal radiation sources.

1. Liquid dispenser comprising a plurality of containers for holding a liquid to be dispensed, a support supporting the containers, and at least one source for biocidal radiation,
wherein each container comprises a lid, which is transparent for the biocidal radiation,
wherein the at least one biocidal radiation source and the plurality of containers are movable relative to each other,
wherein the source for biocidal radiation is at a distance above the lids,
characterized in that the containers are arranged on a turntable in concentric circular arrays.
wherein the turntable is movable relative to the at least one biocidal radiation source, and
wherein the source of biocidal radiation is arranged to irradiate each container during time intervals of essentially the same length.

2. Dispenser according to claim 1, wherein the source for biocidal radiation is arranged to irradiate the surface of a colorant in the container and/or an inner wall of a headspace of the container and/or a stirrer within the container.

3. Dispenser according to claim 1, wherein the biocidal radiation is UV-light.

4. Dispenser according to claim 3, wherein the UV-light is irradiated with an intensity of at least 4 mJ/cm2.

5. Dispenser according to claim 1, wherein the transparent lid is made of a UV-transparent material.

6. (canceled)

7. (canceled)

8. Dispenser according to claim 1, wherein the source of biocidal radiation comprises a plurality of radiation devices, each circular array of containers comprising at least one of said plurality of radiation devices.

9. Dispenser according to claim 8, wherein each radiation device has a width which is proportional with the radius of the respective concentric array.

10. Dispenser according to claim 8, comprising a fixture carrying a plurality of the radiation devices and having one or more openings allowing passage of radiation from the radiation devices to at least one of the containers positioned below the respective opening.

11. Dispenser according to claim 10, wherein the radiation devices are above a single opening shaped as a circular segment coaxial with the turntable.

12. Dispenser according to claim 8, wherein the radiation devices are controlled independently of each other.

13. Dispenser according to claim 1, wherein the source of biocidal radiation comprises one or more mirrors, movable between a plurality of positions in which the mirror deflects a beam from the source of biocidal radiation to a different one of the containers.

14. Dispenser according to claim 13, wherein the source of biocidal radiation is moveable along a guide in radial direction relative to the central axis of rotation of the turntable.

15. Dispenser according to claim 3, wherein the biocidal radiation is UVB and/or UVC light.

16. Dispenser according to claim 3, wherein the biocidal radiation is UV light having a wave length of 100-320 nm.

17. Dispenser according to claim 3, wherein the UV-light is irradiated with an intensity of at least 8 mJ/cm2.

18. Dispenser according to claim 3, wherein the UV-light is irradiated with an intensity of at least 10 mJ/cm2.

19. Dispenser according to claim 3, wherein the UV-light is irradiated with an intensity of at least 12 mJ/cm2.

20. Dispenser according to claim 5, wherein the transparent lid is made from the group of quartz glass, borosilicate glass, polystyrene, polymethyl methacrylate, polycarbonate or fluorinated ethylene propylene (FEP).

21. Dispenser according to claim 5, wherein the transparent lid is made from a copolymer of tetrafluoroethylene and hexafluoropropylene.
